(19) United States
(12) Patent Application Publication
(10) Pub. No.: US 2022/0056059 A1
(43) Pub. Date: Feb. 24, 2022

(54) 6,6'-([1,1'-BIPHENYL]-2,3'-DIYLBIS(OXY))DIDIBENZO[D,F][1,3,2]DIOXAPHOSPHEPINES

(71) Applicant: EVONIK OPERATIONS GMBH, Essen (DE)

(72) Inventors: Robert FRANKE, Marl (DE); Armin BÖRNER, Rostock (DE); Svenja Kloss, Albstadt (DE); Detlef Selent, Rostock (DE)

(73) Assignee: EVONIK OPERATIONS GMBH, Essen (DE)

(21) Appl. No.: 17/402,893

(22) Filed: Aug. 16, 2021

(30) Foreign Application Priority Data
Aug. 18, 2020 (EP) 20191435.5

(51) Int. Cl. C07F 9/6574; C07C 45/50; B01J 31/24

(52) U.S. Cl. CPC C07F 9/65746; C07C 45/505; B01J 2231/321; B01J 2531/822; B01J 31/2471

(57) ABSTRACT

6,6′-([1,1′-Biphenyl]-2,3′,-diylbis)oxy))didibenzo[d,f][1,3,2]dioxaphosphepines and the use thereof in hydroformylation.

The invention relates to 6,6′-([1,1′-biphenyl]-2,3′-diylbis(oxy))didibenzo[d,f][1,3,2]dioxaphosphepines and to the use thereof in hydroformylation.

Phosphorus-containing compounds play a crucial role as ligands in a multitude of reactions, e.g. in hydrogenation, in hydrocyanation and also in hydroformylation.

The reactions between olefin compounds, carbon monoxide and hydrogen in the presence of a catalyst to give the aldehydes with one carbon atom more are known as hydroformylation or the oxo process. Catalysts used in these reactions are frequently compounds of the transition metals of group VIII of the periodic table of the elements. Known ligands are, for example, compounds from the phosphine, phosphite and phosphonite classes, each containing trivalent phosphorus P^{III}. A good overview of the situation on the hydroformylation of olefins can be found in R. Franke, D. Selent, A. Börner. “Applied Hydroformylation”. Chem. Rev., 2012, DOI:10.1021/cr3001803.

In EP 0 577 042 A1, on page 6, the following ligand is described:

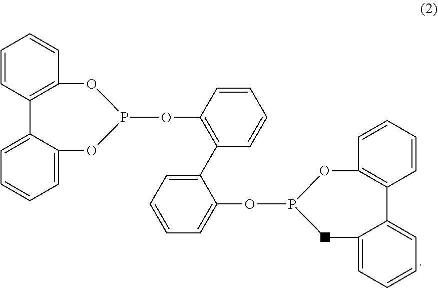 [SMILES: c1ccc2c(c1)CP(Oc1ccccc1-c1ccccc1Op1oc3ccccc3c3ccccc3o1)Oc1ccccc1-2]

The technical object of the invention is to provide new ligands that exhibit increased n/iso selectivity in the hydroformylation of olefins compared with the ligand known from the prior art.

The object is achieved by a compound according to Claim 1.

Compound of the Structure (I):

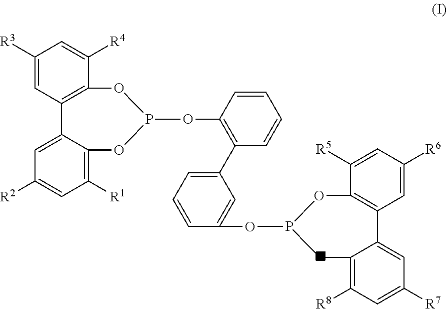

where R¹, R², R³, R⁴, R⁵, R⁵, R⁶, R⁷, R⁸ are selected from: —H, —(C₁-C₁₂) alkyl, —O—(C₁-C₁₂) alkyl.

In one embodiment, R¹, R⁴, R⁵, R⁸ are selected from: —H, —(C₁-C₁₂) alkyl.

In one embodiment, at least one of the radicals R¹, R⁴, R⁵, R⁸ is —H.

In one embodiment, R¹, R⁴, R⁵, R⁶ are —H.

In one embodiment, R², R³, R⁶, R⁷ are selected from: —H, —O13 (C₁-C₁₂) alkyl.

In one embodiment, at least one of ths radicals R², R³, R⁵, R⁷ is —H.

In one embodiment, R², R³, R⁶ R⁷ are 13 H.

In one embodiment, the compound has the structure (1):

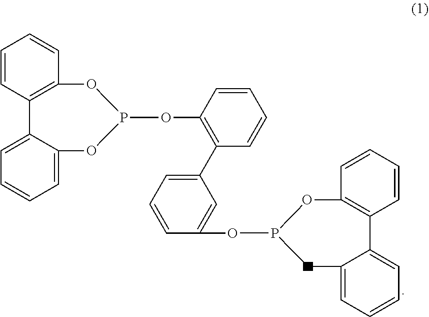 [SMILES: c1cc(OP2Cc3ccccc3-c3ccccc3O2)cc(-c2ccccc2Op2oc3ccccc3c3ccccc3o2)c1]

As well as the compound per se, the use thereof for catalysis of a hydroformylation reaction is also claimed.

Use of an above-described compound in a ligand-metal complex for catalysis of a hydroformylation reaction.

Additionally claimed is a process in which the above-described compound is used as a ligand.

Process comprising the process steps of:

a) initially charging an olefin,

b) adding an above-described compound and a substance containing a metal selected from: Rh, Ru, Co, Ir,

c) feeding in H₂ and CO,

d) heating the reaction mixture from steps a) to c), with conversion of the olefin to an aldehyde.

In a preferred embodiment, the metal is Rh.

The ligands can also be used in excess here and it is not automatically the case that each ligand is present in bound form as a ligand-metal complex; it may instead be present m the reaction mixture as the free ligand.

The reaction is carried out under customary conditions.

Preference is given to a temperature of 80° C. to 160° C. and a pressure of 10 to 60 bar.

Particular preference is given to a temperature of 100° C. to 140° C. and a pressure of 20 to 50 bar.

The reactants for the hydroformylation in the process of the invention are olefins or mixtures of olefins, especially monoolefins having 2 to 24, preferably 3 to 16 and more preferably 3 to 12 carbon atoms, and having terminal or internal C—C double bonds, for example 1-propene, 1-butene, 2-butene, 1- or 2-pentene, 2-methyl-1-butene, 2-methyl-2-butene, 3-methyl-1-butene, 1-, 2- or 3-hexene, the C₆ olefin mixture obtained in the dimerization of propane (dipropene), heptenes, 2- or 3-methyl-1-hexenes, octenes, 2-methylheptenes, 3-methylheptenes, 5-methyl-2 heptane, 6-methyl-2-heptene, 2-ethyl-1-hexene, the C₈ olefin mixture obtained in the dimerization of butenes (di-n-butene, diisobutene), nonenes, 2- or 3-methyloctenes, the C₉ olefin mixture obtained in the trimerization of propene (tripropene), decenes, 2-ethyl-1-octane, dodecenes, the C₁₂ olefin mixture obtained in the tetramenzation of propene or the trimerization of butenes (tetrapropene or tributene), tetradecenes, hexadecenes, the C₁₆ olefin mixture obtained in the tetramerization of butenes (tetrabutene), and olefin mixtures having different numbers of carbon atoms (preferably 2 to 4) produced by cooligomerization of olefins.

The process of the invention using the ligands of the invention can be used for the hydroformylation of α-olefins, terminally branched, internal and internally branched olefins.

The invention shall be illustrated in detail hereinbelow with reference to exemplary embodiments.

Work Procedures

All the preparations that follow were carried out under inert gas using standard Schlenk techniques. The solvents were dried before use over suitable drying agents.

The products were characterized by NMR spectroscopy. Chemical shifts (δ) are reported in ppm. The ³¹P NMR signals were referenced as follows: SR³¹P=SR¹H * (BF³¹P/BF¹H)=SR¹H * 0.4048.

Synthesis of 6,6′-[(1,1′-biphenyl]-2,3′-diylbis(oxy)didibenzo[d,f][1,3,2]dioxaphosphepin (1)

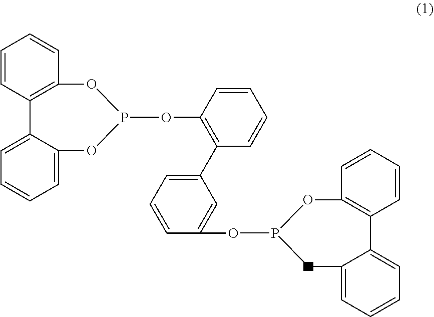 [SMILES: c1cc(OP2Cc3ccccc3-c3ccccc3O2)cc(-c2ccccc2Op2oc3ccccc3c3ccccc3o2)c1]

A solution of 0.10 g of 2,3′-biphenol (0.56 mmol) and 0.3 ml of triethylamine (2.24 mmol) in 4 ml of THF was added dropwise at −20° C. to a stirred solution of 0.39 g of 6-chlorodibenzo[d,f][1,3,2]-dioxaphosphepin (1.56 mmol) in 4 ml of THF. The solution was stirred further and warmed to room temperature overnight. The solvent was then removed under reduced pressure, the residue taken up in 10 ml of toluene and filtered through a G4 frit. The solvent of the filtrate was then removed under reduced pressure. The yellow oil left behind was worked up by column chromatography (eluent mixture dichloromethane/n-heptane=3:7). This afforded 0.10 g of a white solid (yield: 30%).

¹H NMR (300 MHz, CD₂Cl₂): δ (ppm)=7.39-7.53 (m; 6H); 7.18-7.39 (m; 16H); 7.00-7.05 (m; 2H). ¹³C NMR (75 MHz, CD₂Cl₂): δ (ppm)=151.8 (d; J_{CP}=7.9 Hz); 149.2 (d; J_{CP}=5.0 Hz); 149.2 (d; J_{CP}=5.0 Hz); 149.0 (d; J_{CP}=7.6 Hz; C_{Ar}OP); 139.9; (d; J_{CP}=3.2 Hz); 133.3; 131.5; 131.4 (d; J_{CP}=3.3 Hz); 131.3 (d; J_{CP}=3.3 Hz); 130.3; 130.2; 129.9; 129.6; 129.4; 126.3; 125.8; 125.8; 125.1; 122.4; 122.3; 121.4; 121.2; 119.8; 119.6.

³¹P NMR (121 MHz, CDh₂Cl₂): δ (ppm)=144.3 (s); 144.0 (s).

HRMS (ESI): Calculated for C₃₆H₂₄O₆P₂ (M+H⁺) 615.11209, found 615.11174. Calculated for C₃₆H₂₄O₆P₂ (M+Na⁺) 637.09403, found 637.09386.

### Synthesis of 2,2′(bis(dibenzo[d,f][1,3,2]dioxaphosphepin-6-yloxy)-1,1′-biphenyl (2) (comparative ligand)

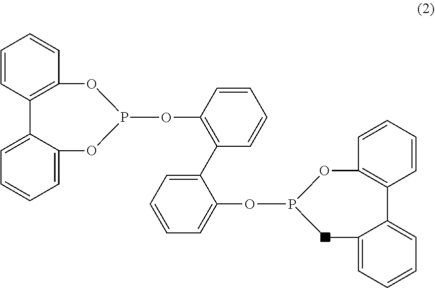 [SMILES: c1ccc2c(c1)CP(Oc1ccccc1-c1ccccc1Op1oc3ccccc3c3ccccc3o1)Oc1ccccc1-2]

A solution of 0.99 g of 2,2′-biphenol (5.29 mmol) and 3 ml of triethylamine (21.2 mmol) in 7 ml of THF was added dropwise at −20° C. to a stirred solution of 2.65 g of 6-chlorodibenzo[d,f][1,3,2]-dioxaphosphepin (10.59 mmol) in 7 ml of THF. The solution was stirred further at room temperature overnight. The solvent was then removed under reduced pressure and the residue taken up in 15 ml of toluene. The turbid solution was filtered through a G4 frit and the residual solvent in the filtrate then removed under reduced pressure. The oily residue was dissolved in a small amount of dichloromethane (approx. 3 ml). n-Heptane was then added with stirring until the solution became turbid. The solution was left overnight in a refrigerator and the clear supernatant solution decanted the next day and the solid dried under reduced pressure. This afforded 1.09 g of a yellowish white solid (yield: 34%). ¹H NMR (300 MHz, CD₂Cl₂): δ (ppm)=7.30-7.49 (m; 11H); 7.23-7.29 (m; 9H); 6.85-6.94 (m; 4H). ¹³C NMR (75 MHz, CD₂Cl₂): δ (ppm)=149.9 (m; C_{Ar}OP); 149.2 (m; C_{Ar}OP); 132.5; 131.3; 130.6; 130.0; 129.5; 129.4; 125.6; 124.6; 122.3; 120.8 (m).

³¹P NMR (121 MHz, CD₂Cl₂): δ (ppm)=144.7 (s).

HRMS (ESI): Calculated for C₃₆H₂₄O₆P₂ (M+H⁺)⁺615.11209, found 615.11203. Calculated for C₃₆H₂₄O₆P₂ (M+Na)⁺637.09403, found 637.09394.

### Catalysis Experiments

The hydroformylation was carried out in a 16 ml autoclave from HEL Group, Hertfordshire, United Kingdom, equipped with a pressure-retaining valve, gas flowmeter and sparging stirrer. The n-octene used as substrate (Oxeno GmbH, mixture of octene isomers composed of 1-octene: 3%; cis+trans-2-octene: 49%; cis+trans-3-octene: 29%; cis+trans-4-octene: 16%; structurally isomeric octenes: 3%) was heated under reflux over sodium for several hours and distilled under argon.

The reaction solutions for the experiments were prepared beforehand under an argon atmosphere. For this, 0.0021 g of Rh(acac)(CO)₂ and the corresponding amount of phosphite compound were weighed out and diluted with 8.0 ml of toluene. The mass of toluene introduced in each case was determined for the GC analysis. 1.80 g of n-octene (16 mmol) was then added. The prepared solutions were then introduced into the autoclave, which was flushed three times with argon and three times with syngas (Linde, H₂ (99.999%):CO (99.997%)=1:1). The autoclave was then heated to the desired temperature at an overall pressure of 10 bar with stirring (900 rpm). On reaching the reaction temperature, the syngas pressure was increased to 20 bar and the reaction carried out at constant pressure for 4 h. At the end of the reaction time, the autoclave was cooled to room temperature, depressurized while stirring and flushed with argon. 0.5 ml of each reaction mixture was removed at the end of the reaction, diluted with 4 ml of pentane and analysed by gas chromatography: HP 5890 Series II plus, PONA, 50 m×0.2 mm×0.5 μm. Residual olefin and aldehyde were quantitatively determined against the solvent toluene as internal standard.

Results of the Catalysis Experiments

Reaction Conditions 1:

[Rh]: 1.0*10⁻³ mol/l, L:Rh=1:2, p: 20 bar, T: 120° C.; t: 4 h

TABLE 1
Hydroformylation of n-octenes

| Ligand | n/iso selectivity in % |
|---|---|
| 1* | 61.6 |
| 2 | 38.9 |

*inventive compound

Reaction Conditions 2:

[Rh]: 1.0*10⁻³ mol/l, L:Rh=1:4, p: 20 bar, T: 120° C.; t: 4 h

TABLE 2
Hydroformylation of n-octenes

| Ligand | n/iso selectivity in % |
|---|---|
| 1* | 78.3 |
| 2 | 68.3 |

*inventive compound

Definition of Selectivity:

In the hydroformylation there is n/iso selectivity, which is the ratio of linear aldehyde (=n) to branched aldehyde (=iso). The selectivity here in respect of the n-aldehyde signifies that this amount of linear product was formed. The remaining percentages then correspond to the branched isomer. Thus, at a regioselectivity of 50%, n-aldehyde and iso-aldehyde are formed in equal proportions.

The compound of the invention (1) achieved an increase in selectivity compared with the comparative ligand (2).

The experiments carried out demonstrate that the stated object is achieved by the compound of the invention.

1. Compound of the structure (I): 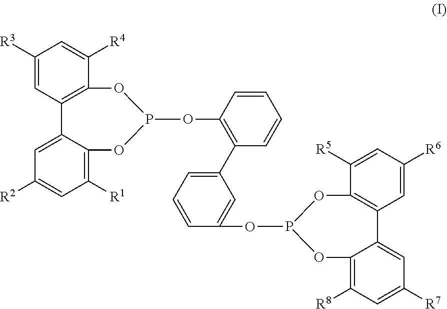
where R¹, R², R³, R⁴, R⁵, R⁶, R⁷, R⁸ are selected from: −H, −(C₁-C₁₂) alkyl, —O—(C₁-C₁₂) alkyl.

2. Compound according to claim 1, where R¹, R⁴, R⁵, R⁸ are selected from: —H, —(C₁-C₁₂) alkyl.

3. Compound according to claim 1, where at least one of the radicals R¹, R⁴, R⁵, R⁸ is —H.

4. Compound according to claim 1, where R¹, R⁴, R⁵, R⁸ are —H.

5. Compound according to claim 1, where R², R³, R⁵, R⁷ are selected from: —H, —O—(C₁-C₁₂) alkyl.

6. Compound according to claim 1, where at least one of the radicals R², R³, R⁶, R⁷ is —H.

7. Compound according to claim 1, where R², R³, R⁶, R⁷ are —H.

8. Compound according to claim 1, where the compound has the structure (1):

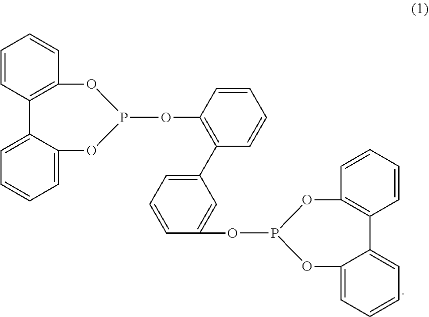 [SMILES: c1cc(Op2oc3ccccc3c3ccccc3o2)cc(-c2ccccc2Op2oc3ccccc3c3ccccc3o2)c1]

9. Use of a compound according to claim 1 in a ligand-metal complex for catalysis of a hydroformylation reaction.

10. Process comprising the process steps of:
a) initially charging an olefin,
b) adding a compound according to claim 1 and a substance containing a metal selected from: Rh, Ru, Co, Ir,
c) feeding in H₂ and CO,
d) heating the reaction mixture from steps a) to c), with conversion of the olefin to an aldehyde.
